(19) United States
(12) Patent Application Publication
(10) Pub. No.: US 2024/0409494 A1
(43) Pub. Date: Dec. 12, 2024

(54) METHOD OF PRODUCING ALKOXYLATED ETHER AMINES AND USES THEREOF

(71) Applicant: HUNTSMAN PETROCHEMICAL LLC, The Woodlands, TX (US)

(72) Inventors: Maxence M. DELUGE, The Woodlands, TX (US); David C. LEWIS, The Woodlands, TX (US)

(21) Appl. No.: 18/697,491

(22) PCT Filed: Oct. 12, 2022
(86) PCT No.: PCT/US2022/046413

§ 371 Date: Apr. 1, 2024

Related U.S. Application Data
(60) Provisional application No. 63/254,552, filed on Oct. 12, 2021.

(51) Int. Cl. C07C 213/02

(52) U.S. Cl. CPC C07C 213/02

(57) ABSTRACT

A process for preparing alkoxylated ether amines including performing an alkoxylation reaction between a Guerbet alcohol and an epoxide in the presence of a catalyst to obtain an alkoxylated alcohol. Performing an amination reaction on the alkoxylated alcohol in the presence of ammonia and hydrogen to form an alkoxylated Guerbet amine.

### CROSS REFERENCE TO RELATED APPLICATIONS

This application claims priority to U.S. Provisional patent application Ser. No. 63/254,552 filed Oct. 12, 2021. The noted applications are incorporated herein by reference.

### FIELD

The present disclosure generally relates to a process for obtaining an alkoxylated ether primary amine. More specifically, the present disclosure relates to a two-step process for producing an alkoxylated ether amine including reacting a primary alcohol with epoxide and aminating the alkoxylated alcohol product with ammonia and hydrogen.

### BACKGROUND

Fatty primary amines as well as alkoxylated alkyl ether primary amines are well known for being excellent building blocks for preparing various products used as emulsifiers, corrosion inhibitors, fuel and lube additives, or agricultural adjuvant. Various polyetheramines can be prepared by reductive amination of alkoxylated aliphatic and aromatic alcohols. Methods for producing fatty primary amines from Guerbet alcohols have been previously discussed. See, e.g., U.S. Pat. No. 5,808,158. However, primary amines produced in such methods tend to lack flexibility for end use. U.S. Pat. Nos. 5,094,667; 5,264,006; 5,298,038; and 6,114,585 disclose methods for producing Guerbet alkyl ether amines aimed at circumventing the issues with the fatty primary amines described above. However, each of the above referenced patents discloses a two-step process involving cyanoethylation or cyanobutylation of the alkoxylated Guerbet alcohol followed by the hydrogenation of the corresponding nitriles.

Despite the state of the art, there is a continuous need for the development of primary amines that allows for extending the carbon chain length and flexible hydrophobicity.

### DETAILED DESCRIPTION

Before explaining aspects of the present disclosure in detail, it is to be understood that the present disclosure is not limited in its application to the details of construction and the arrangement of components or steps or methodologies set forth in the following description. The present disclosure is capable of other embodiments or of being practiced or carried out in various ways. Also, it is to be understood that the phraseology and terminology employed herein is for the purpose of description and should not be regarded as limiting.

Unless otherwise defined herein, technical terms used in connection with the present disclosure shall have the meanings that are commonly understood by those having ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference to the extent that they do not contradict the instant disclosure.

All of the compositions and/or methods disclosed herein can be made and executed without undue experimentation in light of the present disclosure. While the compositions and methods of the present disclosure have been described in terms of preferred embodiments, it will be apparent to those having ordinary skill in the art that variations may be applied to the compositions and/or methods and in the steps or sequences of steps of the methods described herein without departing from the concept, spirit, and scope of the present disclosure. All such similar substitutes and modifications apparent to those skilled in the art are deemed to be within the spirit, scope, and concept of the present disclosure.

As utilized in accordance with the present disclosure, the following terms, unless otherwise indicated, shall be understood to have the following meanings.

The use of the word “a” or “an”, when used in conjunction with the term “comprising”, “including”, “having”, or “containing” (or variations of such terms) may mean “one”, but it is also consistent with the meaning of “one or more”, “at least one”, and “one or more than one”.

The use of the term “or” is used to mean “and/or” unless clearly indicated to refer solely to alternatives and only if the alternatives are mutually exclusive.

If the specification states a component or feature “may,” “can,” “could,” or “might” be included or have a characteristic, that particular component or feature is not required to be included or have the characteristic.

Throughout this disclosure, the term “about” is used to indicate that a value includes the inherent variation of error for the quantifying device, mechanism, or method, or the inherent variation that exists among the subject(s) to be measured. For example, but not by way of limitation, when the term “about” is used, the designated value to which it refers may vary by plus or minus ten percent, or nine percent, or eight percent, or seven percent, or six percent, or five percent, or four percent, or three percent, or two percent, or one percent, or one or more fractions therebetween.

The use of “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it refers. In addition, the quantities of 100/1000 are not to be considered as limiting since lower or higher limits may also produce satisfactory results.

In addition, the phrase “at least one of X, Y, and Z” will be understood to include X alone, Y alone, and Z alone, as well as any combination of X, Y, and Z. Likewise, the phrase “at least one of X and Y” will be understood to include X alone, Y alone, as well as any combination of X and Y. Additionally, it is to be understood that the phrase “at least one of” can be used with any number of components and have the similar meanings as set forth above.

The use of ordinal number terminology (i.e., “first”, “second”, “third”, “fourth”, etc.) is solely for the purpose of differentiating between two or more items and, unless otherwise stated, is not meant to imply any sequence or order or importance to one item over another or any order of addition.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The phrases “or combinations thereof” and “and combinations thereof” as used herein refers to all permutations and combinations of the listed items preceding the term. For example, “A, B, C, or combinations thereof” is intended to include at least one of: A, B, C, AB, AC, BC, or ABC and, if order is important in a particular context, also BA, CA, CB, CBA, BCA, ACB, BAC, or CAB. Continuing with this example, expressly included are combinations that contain repeats of one or more items or terms such as BB, AAA, CC, AABB, AACC, ABCCCC, CBBAAA, CABBB, and so forth. The skilled artisan will understand that typically there is no limit on the number of items or terms in any combination, unless otherwise apparent from the context. In the same light, the term “and combinations thereof” when used with the phrase “selected from the group consisting of” refers to all permutations and combinations of the listed items preceding the phrase.

The phrases “in one example”, “in an example”, “according to one example”, and the like generally mean the particular feature, structure, or characteristic following the phrase is included in at least one example of the present disclosure, and may be included in more than one example of the present disclosure. Importantly, such phrases are non-limiting and do not necessarily refer to the same example but, of course, can refer to one or more preceding and/or succeeding examples. For example, in the appended claims, any of the claimed examples can be used in any combination.

As used herein, the terms “% by weight”, “wt %”, “weight percentage”, or “percentage by weight” are used interchangeably.

As used herein, the term “ambient temperature” refers to the temperature of the surrounding work environment (e.g., the temperature of the area, building or room where the curable composition is used), exclusive of any temperature changes that occur as a result of the direct application of heat to the curable composition to facilitate curing. The ambient temperature is typically between about 10° C. and about 30° C., more specifically about 15° C. and about 25° C.

As used herein, a “surfactant” refers to a chemical compound that lowers the interfacial tension between two liquids.

A process for synthesizing alkoxylated amines as described herein can be performed using the following two-step process: performing an alkoxylation reaction between (i) a primary alcohol and (ii) an epoxide, then performing a reductive amination of the alkoxylated alcohol via reaction with (iii) ammonia and (iv) hydrogen.

In at least one example, the primary alcohol (i) of the first step of the two-step process can be a saturated primary alcohol having from about 12 to about 40 carbon atoms. In at least one example, the primary alcohol can be a Guerbet alcohol. The term “Guerbet alcohol” as used herein refers branched saturated primary alcohols. Such Guerbet alcohols can provide good lubricity, a high fluidity range, a low melting point, and a high boiling point. For example, the melting point of a Guerbet alcohol can be from about 50° C. to about 60° C. lower than a linear saturated alcohol having the same number of carbons. Additionally, Guerbet alcohols having up to 24 carbon atoms can be liquids at ambient temperature and the alkoxylated surfactants derived from Guerbet alcohols can exhibit a lower viscosity than those prepared from linear saturated alcohols having a corresponding number of carbon atoms. The Guerbet alcohol can include, without limitation, alcohols having a 2-alkyl-1-alkanol general structure.

The epoxide (ii) of the first step can be any epoxide including, without limitation, ethylene oxide, propylene oxide, butylene oxide, pentylene oxide, and styrene oxide. The reaction of primary alcohol with the epoxide can generate an alkoxylated primary alcohol, including, without limitation, an ethoxylated-, propoxylated-, butoxylated-, and pentoxylted-primary alcohol.

The chemical reaction (a) of the first step can be performed in the presence of one or more catalysts. The catalysts used in the first step of the reaction can be any catalyst suitable to effectuate the reaction including, without limitation alkaline and/or heterogeneous catalysts. In at least one example, the catalyst can be selected from sodium hydroxide (NaOH), potassium hydroxide (KOH), sodium methoxide (NaOMe), potassium methoxide (KOMe), ammonia (NH₃), calcium oxide (CaO), calcium carbonate (CaCO₃), a double metal cyanide (DMC), and combinations thereof. The metal present in the DMC catalyst can include, without limitation, zirconium (Zn(II)), iron (Fe(II), Fe(III)), cobalt (Co(II), Co(III)), chromium (Cr(III)), Iridium (Ir(III)), and combinations thereof.

The alkoxylation reaction can be performed at a temperature less than about 200° C. In at least one example, the alkoxylation reaction can be performed at a temperature of less than about 165° C. In an additional example, the alkoxylation reaction can be performed at a temperature of less than about 140° C. In yet another example, the alkoxylation reaction can have a reaction temperature ranging from about 120° C. to about 125° C.

In the second step, an amination reaction can be performed on the alkoxylated alcohol product of the first step. Specifically, the alkoxylated alcohol product can be reacted with ammonia (NH₃) and hydrogen (H₂) to obtain an alkoxylated alcohol amine. In at least one example, the amination reaction can be performed in a batch process. In the alternative, the amination reaction can be performed as a continuous reaction. The continuous process can circumvent an extra filtration step which is required during batch process amination. Additionally, excess ammonia produced during the reaction can be recycled and reused in further continuous processing.

During the amination reaction, the flow rate of the alkyoxylated primary alcohol can enter the reaction chamber at a rate of greater than about 0.5 space velocity. As used herein, the term “space velocity” refers to the relation between volumetric flow and reactor volume in a chemical reactor. In at least one example, the flow rate of the alkoxylated primary alcohol into the reaction chamber can be from about 0.5 space velocity to about 1.5 space velocity. In an alternative example, the flow rate can be from about 0.5 space velocity to about 1.0 space velocity. The ratio of the ammonia to hydrogen flow rate can be from about 0.05 liter per hour (L/hr): 1 gram (g) NH₃/h to 0.12 L/hr: 1 g NH₃/h to 0.65 L/hr: 1 g NH₃/h. The molar ratio of ammonia to alkoxylated primary alcohol in the amination reaction can range from about 8:1 NH₃ to alcohol to about 100:1 NH₃ to alcohol. In at least one example, the molar ratio can range from about 20:1 NH₃ to alcohol to about 50:1 NH₃ to alcohol.

To accelerate the amination of the alkoxylated alcohol, the reaction can be performed in the presence of a catalyst. In at least one example, the catalyst present in amination reaction can be a metal catalyst or a mixture of metal catalysts. Metal catalysts that can be present in the amination reaction can be one or more metals including, without limitation, cobalt, copper, iridium, nickel, rhodium, ruthenium, zirconium, and/or oxides thereof. In at least one example, the catalyst may be supported on a bed of silica, alumina, or graphite. In at least one example, the activity of the catalyst may be increased by activation with hydrogen prior to being introduced into the amination reaction.

In at least one example, the amination reaction can be performed under pressure. In at least one example, the amination reaction can be performed at a temperature greater than about 100° C. In an alternative example, the amination reaction can be performed at a temperature greater than about 130° C. In yet another alternative example, the amination reaction can be performed at a temperature ranging from about 150° C. to about 250° C.

The pressure at which the amination reaction occurs at a range of from about 1500 psig to about 2500 psig. In at least one example, the reaction pressure is from about 1800 psig to about 2000 psig. The temperature of the reaction can range from about 150° C. to 250° C. In at least one example, the temperature can range from about 180° C. to about 220° C.

The amination process described herein can allow for a high conversion of alcohol to amine. For example, the amination reaction process described herein can produce a higher selectivity towards primary amine compared to previous processes involving cyanoethylation followed by hydrogenation.

The alkoxylated amines formed using the reaction described herein can be used in a variety of markets including, without limitation, agrochemicals, coatings, adhesives, industrial markets, gas treatments, electronics, construction, composites, metalworking, mining, oil field chemicals, enhanced oil recovery, paper, polyurethane additives, polyurethane components, water, fuels, lubricants, and polymer modification.

Additionally, the alkoxylated amines can be used in various applications including, without limitation, home and personal care applications (including, without limitation, soaps, detergents, etc.), asphalt emulsifiers (as the acetate salt of Di C10+), dispersants (e.g., reacted into dispersant components), as a reagent for iron ore (e.g., Taconite) reverse flotation, reacted into polymers to provide hydrophobic side chains (e.g., C20-40 amine reacted into maleic to make a paraffin inhibitor), as an additive for various plastics (e.g., internal mold release); and the like.

The alkoxylated amines can be further used as initiators for alkoxylates including, without limitation, C14-40 Guerbet amines and ethylene oxide/propylene oxide, or ethylene oxide/propylene oxide carboxylated, or ethylene oxide/propylene oxide sulfamated-ultra low IFT surfactants for EOR; groundwater remediation (e.g., removal of NAPL non-aromatic polluting liquids); C12-24 and ethoxylated for agricultural formulations (e.g., chemicals like Round up-type (SL) or in EC formulations); octyl/decanol amine and ethoxylated as a replacement for oxtyl phenol ethoxylates; a foaming agent for urethane foams; an anti-redeposition aid in detergents, a fuel lubricant (e.g., friction modifier), and the like.

Examples are provided below. However, the present disclosure is to be understood to not be limited in its application to the specific experiments, results, and laboratory procedures disclosed herein below. Rather, the Examples are simply provided as one of various embodiments and are meant to be exemplary and not exhaustive.

### EXAMPLES

To exemplify the two-step reaction described herein, the propoxylation of a Guerbet alcohol to form a propoxylated Guerbet amine product is illustrated below:

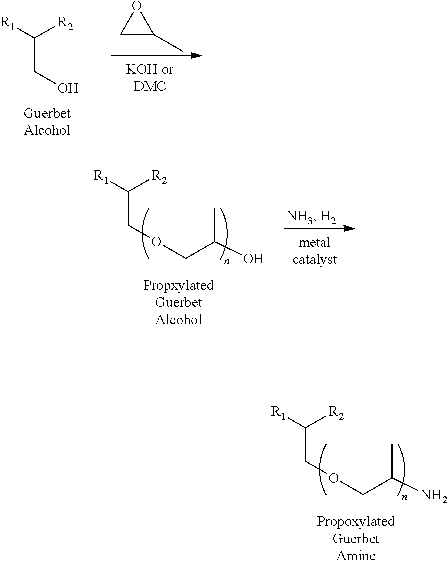

where R1 and R2 are each individually be selected from saturated alkyl chains having x and y integer number of carbons, where x+y ranges from 14 to 36; DMC is a double metal cyanide catalyst, as described in detail above; and n is an integer between 2 and 35.

The term “alkyl” as used herein is inclusive of both straight chain and branched chain groups and of cyclic groups. In some examples, alkyl groups R1 and R2 may have up to 40 carbons (in some embodiments up to about 30, 20, 15, 12, 10, 8, 7, 6, 5, 4, 3, 2, or 1 carbons) unless otherwise specified. In at least one example, cyclic alkyl groups compatible with the alkyl chains described above can be monocyclic and can have from about 3 to about 10 carbon atoms.

The exemplary propoxylation reaction above can include charging a Guerbet alcohol in a stainless steel kettle in the presence of a catalyst. The Guerbet alcohol can include from about 12 to about 40 carbon atoms. After which, the reaction temperature may be raised to about 120° C. and the resulting mixture can be dried under the flow of nitrogen. Then, from about 2 equivalents to about 35 equivalents of propylene oxide can be added while the reaction pressure is maintained at about 60 psi and a temperature of from about 120° C. to about 125° C. During the reaction, the temperature can be maintained at a range of from about 120° C. to about 125° C. as the pressure within the reaction chamber fluctuates. The pressure can be adjusted by varying the flow of propylene oxide into the reaction chamber. Once the pressure drops below about 1 psi for a period of at least about 30 minutes, the mixture can be cooled and analyzed for hydroxyl number.

The second step of the exemplary reaction above includes reacting the propoxylated Guerbet alcohol with a flow of hydrogen (H₂) and ammonia (NH₃), as described above. The flow of hydrogen can be fluctuated to adjust the reaction pressure within a reaction chamber to a desired pressure. In at least one example, the desired pressure can be in the range of about 1800 psig and about 2000 psig. The flow of hydrogen can then be further adjusted to achieve a desired flow rate between about 2.6 L/hr to about 3.2 L/hr. The flow of ammonia can enter the reaction chamber at a rate of about 50 g/hr.

A propoxylated Guerbet alcohol flow can enter the reaction chamber at a rate from about 50 g/hr to about 100 g/hr. In at least one example, the propoxylated Guerbet alcohol flow rate can be from about 50 g/hr to about 75 g/hr. After about 1 hour to about 2 hours of reaction time, and when a steady state has been achieved, a reaction product can be collected and tested for an amine amount.

When a catalyst is present, the catalyst may be activated by the hydrogen flow prior to entering the reaction chamber to accelerate the amination reaction. For example, 100 g of a metal catalyst can be charged into a 100 mL stainless steel continuous reactor and activated under the flow of hydrogen at a rate of about 50 L/hr for a period of about 2 hours and a temperature of about 200° C. The temperature of the reaction chamber can then be adjusted to a desired range. In at least one example, the desired temperature range can be from about 180° C. to about 220° C. In an additional example, the desired temperature range can be from about 200° C. to about 210° C.

In some examples, after the reaction is completed, some amount of crude product can remain. In at least one example, the remaining crude product can be charged in a glass reactor, and excess ammonia and a water by-product can be stripped under vacuum at a temperature of about 120° C. The purified crude product can then be recycled to the beginning of the reaction.

Specific examples of the exemplary propoxylation and amination reaction are provided below. Such examples are not all-inclusive and are not intended to be limiting or limit the scope of the disclosure herein.

### Procedure for Propoxylation of Guerbet Alcohols

A clean and dry 4-gallon stainless steel kettle is loaded with the Guerbet alcohol and the catalyst. When the catalyst is 45% potassium hydroxide (KOH) a 6 mol percent (mol %) can be used. When the catalyst is DMC 0.06 wt % can be used. The reaction temperature is then raised to 120° C. with a nitrogen sparge at 6 to 8 scfh. The reaction is maintained at such conditions for a period of 2 hours to remove traces of water. Subsequently, 2 equivalents to 35 equivalents of propylene oxide is added while maintaining the reaction pressure below 60 psi and the reaction temperature at 120° C. to 125° C. The reaction temperature is then held at 120-125° C. until the pressure drop is below 1 psi for a period of 30 minutes. The remaining pressure is then vented to a scrubber and the residual, unreacted propylene oxide is removed by sparging nitrogen at about 6 scfh for a period of 30 minutes. The mixture is then cooled down and analyzed to determine hydroxyl number.

### Procedure for Amination of Propoxylated Guerbet Alcohols

A clean and dry 100 mL stainless steel continuous tubular reactor is loaded with about 100 g of a metal catalyst. The metal catalyst can be a metal or a mixture of metals including, but not limited to cobalt, copper, iridium, nickel, rhodium, ruthenium, zirconium, and/or oxides thereof. Hydrogen is flowed through the reactor at a rate of 50 L/h for a period of 2 hours at a temperature of 200° C. to activate the catalyst. The reaction temperature is then increased to 205° C. and the pressure is increased to 2000 psig with hydrogen. The hydrogen flow is then set to 3.1 L/h. An ammonia flow enters the reaction at a rate of 50 g/h. A flow of propoxylated Guerbet alcohol enters the reaction at a flow rate of 75 g/h. After a period of 2 hours, a steady state is achieved and the product can be collected and analyzed for amine count. After the reaction, the crude product is charged in a glass reactor and the excess ammonia and the water by-product are stripped under a vacuum at 120° C.

The following comparative and experimental examples were performed to evaluate the methods described herein:

### Example 1 (“E1”): Guerbet C16 and 5 Propylene Oxide Amine

Following the procedures for propoxylation as set out above, Isofol 16 was reacted with 5 equivalents of propylene oxide in the presence of DMC. Subsequently, the Guerbet+5PO alcohol obtained was aminated in accordance with the procedure described above. Tests were performed after the propoxylation and amination steps to determine hydroxyl and amine numbers, results of which are provided in Table 1.

### Example 2 (“E2”): Guerbet C16 and 10 Propylene Oxide Amine

The same method as described for Example 1 was used except that Isofol 16 was reacted with 10 equivalents of propylene oxide in the presence of a potassium hydroxide (KOH) catalyst. Subsequently, the propoxylated alcohol was aminated as described above. Tests were performed after each step to determine hydroxyl and amine numbers, results of which are provided in Table 1.

### Example 3 (“E3”): Guerbet C16 and 15 Propylene Oxide Amine

The same method as described for Example 1 was used except that Isofol 16 was reacted with 15 equivalents of propylene oxide in the presence of DMC. The propoxylated alcohol was then aminated as described above. Tests were performed after each step to determine hydroxyl and amine numbers, results of which are provided in Table 1.

### Example 4 (“E4”): Guerbet C18T and 5 Propylene Oxide Amine

The same method as described for Example 1 was used except that Isofol 18T was reacted with 5 equivalents of propylene oxide in the presence of a DMC catalyst. The amination was then performed as indicated above. Tests were performed after each step to determine hydroxyl and amine numbers, results of which are provided in Table 1.

### Example 5 (“E5”): Guerbet C20 and 5 Propylene Oxide Amine

The same method as described for Example 1 was used except that Isofol 20 was reacted with 5 equivalents of propylene oxide in the presence of a DMC catalyst. The amination of the propoxylated Guerbet alcohol is performed as indicated above. Tests were performed after each step to determine hydroxyl and amine numbers, results of which are provided in Table 1.

### Example 6 (“E6”): Guerbet C28 and 5 Propylene Oxide Amine

The same method as described for Example 1 was used except that Jarcol 28 was reacted with 5 equivalents of propylene oxide in the presence of a DMC catalyst. The amination of the propoxylated Guerbet alcohol is performed as indicated above. Tests were performed after each step to determine hydroxyl and amine numbers, results of which are provided in Table 1.

TABLE 1

| | | | OH | Total | | |
|---|---|---|---|---|---|---|
| | | | Number | Amine | | |
| | Guerbet | PO | (mg | (mg | Primary | Secondary |
| | Alcohol | Addition | KOH/g) | KOH/g) | Amine (%) | Amine (%) |
| E1 | C16 | 5 | 108.2 | 100.7 | 98.2 | 1.8 |
| E2 | C16 | 10 | 63.2 | 65.7 | 99.4 | 0.6 |
| E3 | C16 | 15 | 44.9 | 41.9 | 98.9 | 1.1 |
| E4 | C18 | 5 | 97.4 | 94.9 | 98.1 | 1.9 |
| E5 | C20 | 5 | 102.6 | 98.9 | 98.3 | 1.7 |
| E6 | C28 | 5 | 79.0 | 75.6 | 98.5 | 1.5 |

The conversion of Guerbet alcohol to propoxylated amine is higher than 92% and the selectivity is higher than 98% towards the formation of the primary amines.

From the above description, it is clear that the present disclosure is well adapted to carry out the object and to attain the advantages mentioned herein as well as those inherent in the present disclosure. While exemplary embodiments of the present disclosure have been described for the purposes of the disclosure, it will be understood that numerous changes may be made which will readily suggest themselves to those skilled in the art which can be accomplished without departing from the scope of the present disclosure and the appended claims.

1. A process for preparing alkoxylated ether amines comprising:
alkoxylating an alcohol with an epoxide in the presence of a catalyst to form an alkoxylated alcohol, wherein the alcohol is a Guerbet alcohol; and
aminating the alkoxylated alcohol in the presence of ammonia and hydrogen to form an alkoxylated Guerbet amine.

2. The process of claim 1, wherein the Guerbet alcohol has the following structure: 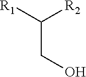
wherein R₁ and R₂ are selected from saturated alkyl chains having x and y integer number of carbons, and wherein x+y ranges from about 14 to about 36.

3. The process of claim 1, wherein the epoxide is selected from ethylene oxide, propylene oxide, butylene oxide, pentylene oxide, and styrene oxide.

4. The process of claim 3, wherein the epoxide is propylene oxide.

5. The process of claim 1, further comprising varying an epoxide flow rate during the alkoxylation.

6. The process of claim 1, wherein the Guerbet alcohol includes from about 12 to about 40 carbon atoms.

7. The process of claim 1, wherein the alkoxylation reaction is performed in the presence of one or more catalysts.

8. The process of claim 7, wherein the one or more catalysts is selected from sodium hydroxide (NaOH), potassium hydroxide (KOH), sodium methoxide (NaOMe), potassium methoxide (KOMe), ammonia (NH₃), calcium oxide (CaO), calcium carbonate (CaCO₃), a double metal cyanide (DMC), and combinations thereof.

9. The process of claim 1, wherein the alkoxylation is performed at a temperature less than about 200° C.

10. The process of claim 1, wherein the amination is performed as a batch reaction.

11. The process of claim 1, wherein the amination is performed as a continuous reaction.

12. The process of claim 1, wherein the amination is performed in a reaction chamber and the alkyoxylated primary alcohol enters the reaction chamber at a rate of greater than about 0.5 space velocity.

13. The process of claim 1, wherein the molar ratio of ammonia to alkoxylated primary alcohol during the amination ranges from about 8:1 NH₃ to alcohol to about 100:1 NH₃ to alcohol.

14. The process of claim 1, wherein the amination is performed in the presence of a catalyst.

15. The process of claim 14, wherein the catalyst is a metal catalyst or a mixture of metal catalysts.

16. The process of claim 1, wherein the amination is performed at a temperature greater than about 100° C. and at a pressure of from about 1500 psig to about 2500 psig.

17. An alkoxylated Guerbet amine obtained by alkoxylating a Guerbet alcohol with an epoxide in the presence of a catalyst to form an alkoxylated Guerbet alcohol; and aminating the alkoxylated Guerbet alcohol in the presence of ammonia and hydrogen to form the alkoxylated Guerbet amine.

18. The alkoxylated Guerbet amine of claim 17, wherein the epoxide is propylene oxide.

19. The alkoxylated Guerbet amine of claim 18, wherein conversion of the Guerbet alcohol to propoxylated amine is higher than 92% and selectivity is higher than 98% towards formation of a primary amine.

20. The alkoxylated Guerbet amine of claim 17, wherein the Guerbet alcohol has the following structure: 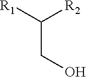
wherein R₁ and R₂ are selected from saturated alkyl chains having x and y integer number of carbons, and wherein x+y ranges from about 14 to about 36.
